(19) United States
(12) Patent Application Publication
(10) Pub. No.: US 2015/0309029 A1
(43) Pub. Date: Oct. 29, 2015

(54) Methods for Cancer Diagnosis, Anti-Cancer Drug Screening, and Test of Drug Effectiveness on the Basis of Phoshorylation of Ras at Thr-144 and Thr-148

(71) Applicant: Industry-Academic Cooperation Foundation, Yonsei University, Seoul (KR)

(72) Inventors: Kang-Yell CHOI, Seoul (KR); Woo-Jeong JEONG, Seoul (KR)

(21) Appl. No.: 14/624,192

(22) Filed: Feb. 17, 2015

Related U.S. Application Data
(63) Continuation of application No. 13/019,270, filed on Feb. 1, 2011, now abandoned.

(30) Foreign Application Priority Data
Feb. 3, 2010 (KR) 10-2010-0010180

(51) Int. Cl. G01N 33/574; C07K 16/40

(52) U.S. Cl. CPC G01N 33/5748; C07K 16/40; G01N 2333/914; C07K 2317/34

(57) ABSTRACT

Methods of diagnosing cancer and screening for an anti-cancer drug using Ras are provided. Ras has a very significant role as a prevalent proto-oncogene which has abnormalities in most forms of cancer, and thus the methods of diagnosing cancer and screening for an anti-cancer drug using Ras may be applied to various forms of cancer. The generation of various forms of cancer in the early stages may be determined by examining whether or not phosphorylation of Ras occurs at Thr-144 and Thr-148 sites. By such a mechanism, an anti-cancer drug having excellent anti-cancer effectiveness may be screened, or the effectiveness of the anti-cancer drug may be tested.

### RELATED APPLICATIONS

This application is a continuation of U.S. application Ser. No. 13/019,270, filed Feb. 1, 2011, entitled “Methods for Cancer Diagnosis, Anti-Cancer Drug Screening, and Test of Drug Effectiveness on the Basis of Phoshorylation of Ras at Thr-144 and Thr-148”; U.S. application Ser. No. 13/019,270 claims priority to and the benefit of Korean Patent application No. 10-2010-0010180 filed Feb. 3, 2010, each of which is incorporated herein by reference in its entirety.

### TECHNICAL FIELD

The present invention relates to a method of diagnosing cancer using Ras, and a method of screening for an anti-cancer drug.

Incorporated by reference herein in its entirety is the Sequence Listing entitled “Sequence_Listing_{—}033864 US_ST25,” created Jun. 20, 2014, size of 4 kilobytes.

### BACKGROUND

Ras proteins bind to guanine nucleotides controlled in activity by conversion of GDP to GTP or vice versa (I. R. Vetter, A. Wittinghofer, Science 294, 1299-1304 (2001)). The abnormal activity of Ras caused by non-hydrolytic GTP-binding mutation is an important factor in generating cancer in humans (J. L. Bos, Cancer Res. 49, 4682-2689(1989)), and thus the mutation of Ras may be found in most cancers. H-, N- and K-Ras iso-forms all have high similarity in amino acid sequence, and variety due to a highly-changeable region of a carboxy-terminal determining functional specificity (K. Wennerberg, K. L. Rossman, C. J. Der, J. Cell Sci. 118, 843-846 (2005)). Ras activity is controlled by various mechanisms, which include activity control through substitution in GTP- and GDP-bound states, and lipid binding control determining migration into a cell membrane (S. Schubbert, K. Shannon, G. Bollag, Nat. Cancer Rev. 7, 295-308 (2007)). It is known that Ras protein trafficking is also influenced by mono- and di-ubiquitination (N. Jura, E. Scotto-Lavino, A. Sobczyk, D. Bar-Sagi, Mol. Cell. 21, 679-687 (2006)). However, a mechanism of controlling such ubiquitination and physiological results for cells or individuals according to Ras ubiquitination, are not known.

### SUMMARY OF THE EMBODIMENTS

The present invention is directed to a novel method of diagnosing cancer using a proto-oncogene, Ras, and a method of screening for an anti-cancer drug.

One aspect of the present invention provides a method of analyzing the level of Ras protein. The method consists of examining whether or not phosphorylation of a proto-oncogene, Ras, occurs at Thr-144 and Thr-148 sites.

As disclosed herein, the level of H-Ras in a cell is controlled by proteolysis using a proteasome of poly-ubiquitinated H-Ras, which is dependent on GSK3β. For the poly-ubiquitination of H-Ras to be mediated by β-TrCP, H-Ras should be phosphorylated by GSK3β at Thr-144 and Thr-148 sites. The following examples show that maintaining the protein level of the proto-oncogene, H-Ras, is correlated with cell proliferation and generation of cancer. For example, if the poly-ubiquitination of H-Ras does not occur due to abnormalities such as mutation of the H-Ras at the Thr-144 and Thr-148 sites or mutation of GSK3β or genes of a Wnt/β-catenin signal transduction system, which can control the phosphorylation of H-Ras at the Thr-144 and Thr-148 sites; H-Ras protein is not proteolysed, resulting in the generation of cancer. In contrast, if the phosphorylation of the H-Ras occurs at the Thr-144 and Thr-148 sites, even if mutations (12, 13, 61 mutations) causing cancer occur in Ras, cancer may not be generated. Therefore, cancer may be diagnosed by examining the phosphorylation of H-Ras at the Thr-144 and Thr-148 sites, and analyzing the level of the H-Ras protein. H-Ras is subjected to proteomic control through ubiquitination by E3-ligase, a ubiquitin ligase β-TrCP, in response to a negative Wnt/β-catenin signal. The lysis is induced by binding β-TrCP to H-Ras by Axin and GSK3β. The present invention shows the phosphorylation of Ras at Thr-144 and Thr-148 through GSK3β by sequentially performing examinations of in vitro phosphorylation and LC/MC-MS/MS mechanical analyses. The importance of the GSK3β-mediated control in response to the Wnt/β-catenin signal in the phosphorylation and ubiquitination of H-Ras is proven by post-expressional control of variants both phosphorylated and non-phosphorylated in GSK3β normal cells and knock-out cells. The phosphorylation of H-Ras in response to the Wnt/β-catenin signal was identified from a rabbit polyclonal antibody specifically recognizing phosphorylated H-Ras. The instability of H-Ras through the phosphorylation of GSK3β shows that cell proliferation and transformation induced by oncogenic Ras in which Leu-61 is substituted may also be inhibited by the controls of protein and polyubiquitination through β-TrCP and the Wnt/β-catenin signal transduction system. Furthermore, it is also confirmed that K-Ras and N-Ras, which are different iso-types of H-Ras, conserve a Thr residue to be phosphorylated and lysed through mechanisms of controlling protein and polyubiquitination by β-TrCP.

Whether or not phosphorylation of Ras at Thr-144 and Thr-148 occurs may be examined by a known technique in the art.

In one exemplary embodiment of the present invention, a method of examining the phosphorylation of Ras at Thr-144 and Thr-148 through an antigen-antibody reaction using an antibody specifically recognizing phosphate groups of Thr-144 and Thr-148 of Ras, is provided. The antibody may be a polyclonal or monoclonal antibody. For example, a monoclonal antibody specifically recognizing the phosphate groups of Thr-144 and Thr-148 of Ras may be produced by a conventional monoclonal antibody producing method in the art. When a secondary antibody labeled with a marker is treated with the monoclonal antibody, or a marker is directly conjugated with the monoclonal antibody, the presence of phosphate groups on Thr-144 and Thr-148 of Ras may be easily examined by a marker. For such a marker, a radioisotope, an emitting material, or a color reaction enzyme may be used. For example, when the labeling is performed using a fluorescent protein such as GFP, YFP, RFP or BFP; the phosphorylation of Ras at Thr-144 and Thr-148 sites may be determined by measuring the quantity of the monoclonal antibody through various methods of fluorimetry. Alternatively, the quantitative analysis of the phosphate groups of Thr-144 and Thr-148 of Ras may be performed by treating the monoclonal antibody specifically recognizing the phosphate groups of Thr-144 and Thr-148 with Ras, and by performing a subsequent color reaction using an enzyme-linked immunosorbent assay (ELISA). In this case, the quantitative analysis may be performed by performing a color reaction using a secondary antibody with which an enzyme such as an alkaline phosphatase (AP) or a horseradish peroxidase (HRP) is conjugated and its substrate, or using an AP or HRP enzyme-conjugated monoclonal antibody specifically recognizing phosphate groups of Thr-144 and Thr-148 of Ras.

In one exemplary embodiment, the antibody may be an antibody against a Ras peptide including amino acids 140-152 of Ras, in which Thr-144 and Thr-148 residues are phosphorylated. In the following exemplary embodiment, a peptide (residues 140-152 PYIEpTSAKpTRQGV; SEQ ID NO: 1) in which Thr-144 and Thr-148 of H-Ras were phosphorylated, binding to a keyhole limpet hemocyanin (KLH) carrier protein, was injected into a rabbit, and the rabbit was killed to obtain blood thereof. From the blood, p-Ras antibodies were purified to be used as antibodies capable of detecting the phosphorylation of Ras at Thr-144 and Thr-148 sites.

Another aspect of the present invention provides a composition for diagnosing cancer including an antibody specifically recognizing phosphate groups of Thr-144 and Thr-148 of Ras. The antibody included in the composition for diagnosing cancer may be an antibody against a Ras peptide including amino acids 140-152 of Ras, in which Thr-144 and Thr-148 residues are phosphorylated, but the present invention is not limited thereto. The antibody may be a polyclonal or monoclonal antibody produced by a known method in the art. The composition may include distilled water or a buffer stably maintaining the antibody structure, rather than the antibody. When the composition for diagnosing cancer is used, the quantity of the antibody specifically recognizing Thr-144 and Thr-148 phosphate groups of Ras is analyzed, thereby examining more easily whether or not phosphorylation of Ras occurs at Thr-144 and Thr-148 sites while also analyzing the level of Ras protein.

Still another aspect of the present invention provides a kit for diagnosing cancer including an antibody specifically recognizing phosphate groups of Thr-144 and Thr-148 of Ras. The antibody included in the kit for diagnosing cancer may be an antibody against a Ras peptide including amino acids 140-152 of Ras, in which Thr-144 and Thr-148 residues are phosphorylated, but the present invention is not limited thereto. The antibody may be a polyclonal or monoclonal antibody produced by a known method in the art. The kit for diagnosing cancer may further include a substrate capable of detecting a marker conjugated with the antibody specifically recognizing Thr-144 and Thr-148 phosphate groups of Ras, or a secondary antibody thereof, rather than the antibody. For example, when an AP or HRP enzyme is conjugated as a marker, a substrate thereof may be further included. The kit for diagnosing cancer, for example, may include a monoclonal antibody specifically recognizing the phosphate groups of Thr-144 and Thr-148 of Ras, the monoclonal antibody being adhered to a 96-well plate, and a separately packaged substrate capable of detecting a marker conjugated with the monoclonal antibody or a secondary antibody thereof. In this case, a cell or tissue lysate sample obtained from a subject to be analyzed may be added to a 96-well plate together with the substrate, and the degree of adhesion of the monoclonal antibody may be quantitatively analyzed by expressing the marker, thereby identifying the level of Ras protein and diagnosing cancer.

Yet another aspect of the present invention provides a method of screening for an anti-cancer drug, the method including contacting Ras with a candidate material, examining whether or not phosphorylation of Ras occurs at Thr-144 and Thr-148 sites, and determining whether the candidate material inhibits or stimulates the phosphorylation. According to whether or not the phosphorylation of Ras occurs at Thr-144 and Thr-148 sites, ubiquitination and the level of Ras protein are determined. When the candidate material inhibits phosphorylation, the level of Ras protein increases. Therefore, it is determined that the candidate material is a carcinogenic material generating cancer. In contrast, when the candidate material stimulates phosphorylation, the proteolysis of Ras protein is stimulated. Therefore, it is determined that the candidate material is an anti-cancer drug inhibiting cancer.

In one exemplary embodiment of the present invention, whether the candidate substrate inhibits or stimulates the phosphorylation may be determined through an antigen-antibody reaction using an antibody specifically recognizing the phosphate groups of Thr-144 and Thr-148 of Ras. The antibody has been described above. Whether the candidate material inhibits or stimulates the phosphorylation may be determined by adding the candidate material and the substrate capable of detecting a marker conjugated with the monoclonal antibody specifically recognizing the phosphate groups of Thr-144 and Thr-148 or a secondary antibody thereof to a plate to which the monoclonal antibody is adhered, and performing quantitative analysis for the degree of adhesion of the monoclonal antibody that expresses the marker.

According to the same principle, yet another aspect of the present invention provides a method of screening for an anti-cancer drug, the method including treating a cell or animal, excluding humans, with a candidate material, and examining whether or not phosphorylation of Ras occurs at Thr-144 and Thr-148 sites, and determining whether the candidate material inhibits or stimulates phosphorylation. In the screening method of the present invention, the candidate material may be a separate nucleic acid, protein, other extracts or natural materials, or a compound which is/are suspected as an anti-cancer drug or randomly selected, according to a conventional selecting method.

Yet another aspect of the present invention provides a composition for screening an anti-cancer drug, the composition including an antibody specifically recognizing Thr-144 and Thr-148 of Ras. The composition may include distilled water or a buffer stably maintaining the antibody structure, rather than the antibody. By quantitative analysis of the antibody specifically recognizing Thr-144 and Thr-148 of Ras using the composition, it can be easily examined whether or not phosphorylation of Ras occurs at Thr-144 and Thr-148 sites, and it can be easily analyzed whether the candidate material inhibits or stimulates the phosphorylation.

Yet another aspect of the present invention provides a method of verifying the effectiveness of an anti-cancer drug, the method including treating an animal, excluding humans, with an anti-cancer drug, and examining whether or not phosphorylation of Ras occurs at Thr-144 and Thr-148 sites and determining whether the candidate material inhibits or stimulates phosphorylation. The procedures for examining whether or not the phosphorylation occurs at Thr-144 and Thr-148 sites, and determining whether the candidate material inhibits or stimulates the phosphorylation are the same as described in the method of screening for an anti-cancer drug described above.

Yet another aspect of the present invention provides for an antibody that specifically recognizes phosphate groups of Thr-144 and Thr-148 of Ras. The antibody may be a polyclonal or monoclonal antibody. The monoclonal or polyclonal antibody specifically recognizing the phosphate groups of Thr-144 and Thr-148 of H-Ras may be produced by a conventional method of producing a monoclonal or polyclonal antibody, which is known in the art. In one exemplary embodiment, the antibody may be an antibody against a Ras peptide including amino acids 140-152 of Ras, in which Thr-144 and Thr-148 residues are phosphorylated.

### Advantageous Effects

Ras is prevelant in almost all forms of cancer as a proto-oncogene. Therefore, methods of diagnosing cancer and screening for an anti-cancer drug using Ras may be applied to various forms of cancer. According to the present invention, the generation of various forms of cancer in the early stages can be determined by examining whether or not phosphorylation of Ras occurs at Thr-144 and Thr-148 sites, and an anti-cancer drug exhibiting excellent effectiveness can be screened or tested in effectiveness by such a mechanism.

### BRIEF DESCRIPTION OF THE DRAWINGS

FIG. 1 shows immunoblotting results identifying that polyubiquitination of H-Ras is increased by GSK3β (FIG. 1A) and by β-TrCP and Axin (FIG. 1B), adversely shows that polyubiquitination is decreased by siRNAs of GSK3β and Axin; and shows axin expression effect on GSK3β-Ras expression (FIG. 1C).

FIG. 2 shows the phosphorylation of H-Ras fused with a purified His tag (His-H-Ras) by GSK3β through an in vitro phosphorylation test (FIG. 2A), and the presence of a conservative sequence for GSK3β-mediated phosphorylation in H-Ras (FIG. 2B);

FIG. 3 shows phosphorylation of H-Ras at Thr-144 and Thr-148 by GSK3β, which is analyzed through liquid chromatography/mass spectrometry (LC-tandam MS/MS) performed by separating H-Ras in which the phosphorylation occurs;

FIG. 4 shows immunoblotting results showing H-Ras control mediated by β-TrCP (FIGS. 4A and 4B) and cytological results (FIG. 4C) in GSK3β normal cells and knock-out cells having a wild-type H-Ras, an activated (cancer-generated) H-Ras by substitution of Leu-61, variants in which Thr residues are substituted with glutamic acids such that T144/T148 residues are phosphorylated, or variants in which Thr residues are substituted with alanines such that T144/T148 residues are not phosphorylated.

FIG. 5 shows immunoblotting results showing that H-Ras proteolysis (FIG. 5A) and polyubiquitination (FIG. 5B) are stimulated by β-TrCP, but are inhibited by Wnt3a in wild-type H-Ras and the variants in which the T144/T148 residues are phosphorylated.

FIG. 6 shows immunoblotting results using the same antibody to show that an antibody recognizing phosphorylated H-Ras protein does not detect non-phosphorylated variant H-Ras, but specifically detects the phosphorylated H-Ras (FIG. 6A), and the phosphorylation of H-Ras is increased by GSK3β (FIG. 6B).

FIG. 7 shows that cell proliferation (FIG. 7A) or transformation (FIGS. 7B, 7C) is not controlled by β-TrCP or Axin when the variants in which the T144/T148 residues can not be phosphorylated are further introduced into H-Ras activated by the substation of Leu-61.

FIG. 8 shows immunoblotting results showing that polyubiquitination of Ras protein overexpressed by treating ALLN (FIG. 8A) is increased, and the quantity of protein is decreased by β-TrCP (FIG. 8B) in K-Ras and N-Ras, like H-Ras.

FIG. 9 shows immunoblotting results where H-, K-, and N-Ras proteins are increased in quantity in GSK3β-knock-out cells as compared to GSK3β-normal cells (FIG. 9A), and an amino acid sequence phosphorylated by GSK3β in the H-Ras is also conserved in K- and N-Ras (FIG. 9B).

The advantages and features of the present invention and the method of revealing them will be explicit from the following examples described in detail. However, it is to be distinctly understood that the present invention is not limited thereto but may be otherwise variously embodied and practiced. It is obvious that the following examples are to complete the disclosure of the invention and to indicate the scope of the present invention to a skilled artisan completely, and the present invention will be defined only by the scope of the claims.

### EXAMPLES

### Example 1

### Polyubiquitination of Ras by Gsk3β

To test for Gsk3β-mediated H-Ras degradation, the levels and polyubiquitination of endogenous Pan-Ras were observed in human embryonic kidney (HEK) 293 cells by small interfering RNA (siRNA)-mediated Gsk3β knockdown and overexpression of Gsk3β. Full-length human H-Ras was isolated from a HEK293 cell cDNA library by PCR and then inserted into the pcDNA-3.1-myc vector using EcoRI and HindIII restriction sites. PcDNA3.1-Gsk3β-V5 was constructed in a similar method. HEK293 cells were transfected with vector/nonspecific siRNA, Gsk3β siRNA, or pcDNA3.1-Gsk3β-V5 together with pCS4-3xFlag-Ub for 24 hours and treated with the proteasome inhibitor ALLN for 12 hours. WCEs (whole cell extracts) were then immunoprecipitated (IP) with anti-Pan-Ras antibody to detect the polyubiquitinated H-Ras. WCEs were immunoblotted with antibodies to Gsk3β, Pan-Ras, Myc-H-Ras or α-tubulin. The siRNA sequences for Gsk3β NM_{—}002093) were 5′-CACUGAUUAUACCUCUAGU-3′ (SEQ ID NO: 2) and 5′-CACUGUAACAUAGUCCGAU-3′ (SEQ ID NO: 3). The GFP siRNA used for the negative control was 5′-GUUCAGCGUGUCCGGCGAGTT-3′ (SEQ ID NO: 4) (synthesized by Samchully Pharmaceutical, Korea).

Furthermore, Gsk3β+/+ or Gsk3β−/− cells were transfected with pCS2-MT-Axin and/or pcDNA3.1-Flag-β-TrCP together with pcDNA3.1-H-Ras and pCS4-3xHA-Ub for 24 hours and then treated with ALLN for 12 hours.

Next, HEK293 cells were transfected with vector/nonspecific siRNA, pSUPER-Axin siRNA, pCS2-MT-Axin, siGsk3β, and/or pcDNA3.1-Gsk3β-V5 together with pcDNA3.1-H-Ras and pCS4-3xFlag-Ub for 24 hours and then treated with ALLN for 12 hours. IP and immunoblotting were performed as described above.

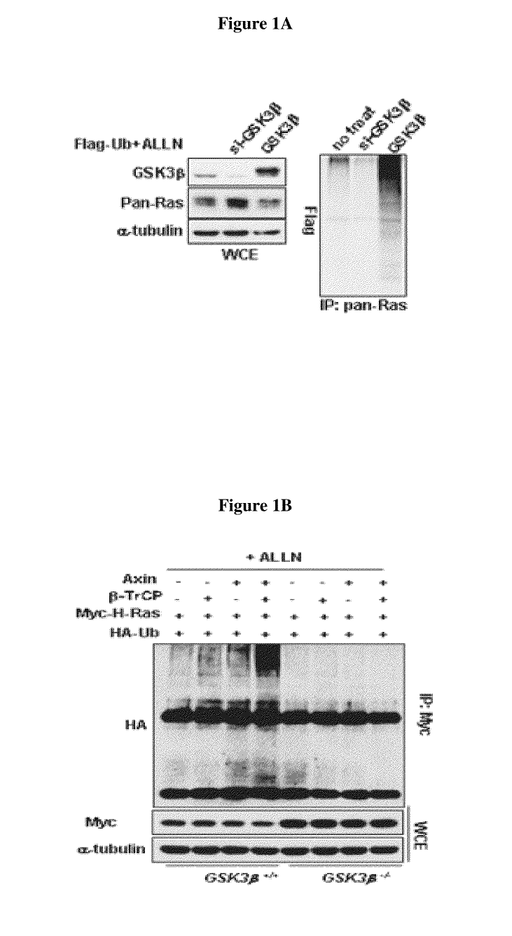

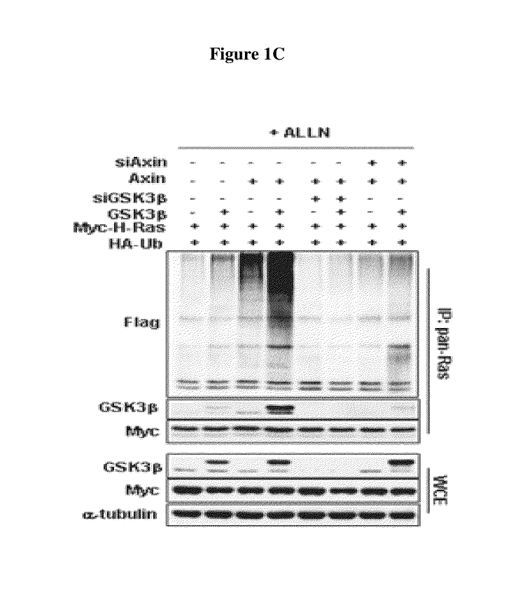

Overexpression or siRNA mediated knockdown of Gsk3β and Axin shows the effect on the polyubiquitination of H-Ras (FIG. 1). The levels of Pan-Ras polyubiquitination were decreased and increased by the knockdown and overexpression of Gsk3β (FIG. 1A). This is evidence that Ras polyubiquitination is mediated by Gsk3β. Based on this result, Gsk3+/+ or Gsk3β−/− cells were used to test the effects of the Axin and β-TrCP overexpression in H-Ras polyubiquitination. In Gsk3β+/+ cells, polyubiquitination of H-Ras was observed due to β-TrCP or Axin expression, but these effects were not observed in Gsk3β−/− cells (FIG. 1B). These results demonstrate that the role of Axin and β-TrCP on Ras protein stability regulation is mediated by Gsk3β. To further understand the role of Axin, H-Ras polyubiquitination was monitored in conditions of overexpression or knockdown of Axin. H-Ras polyubiquitination increased by overexpression of Gsk3β and Axin and decreased by each siRNA consistently. We also found that the Gsk3β-Ras interaction was affected by Axin. Axin overexpression or knockdown enhanced or reduced Gsk3β-Ras interaction (FIG. 1C). This result reflects an important role for Axin in the Gsk3β-dependent regulation of H-Ras protein stability, phosphorylation and polyubiquitination through the modulation of the Gsk3β-Ras interaction.

### Example 2

### Phosphorylation of Ras by Gsk3β

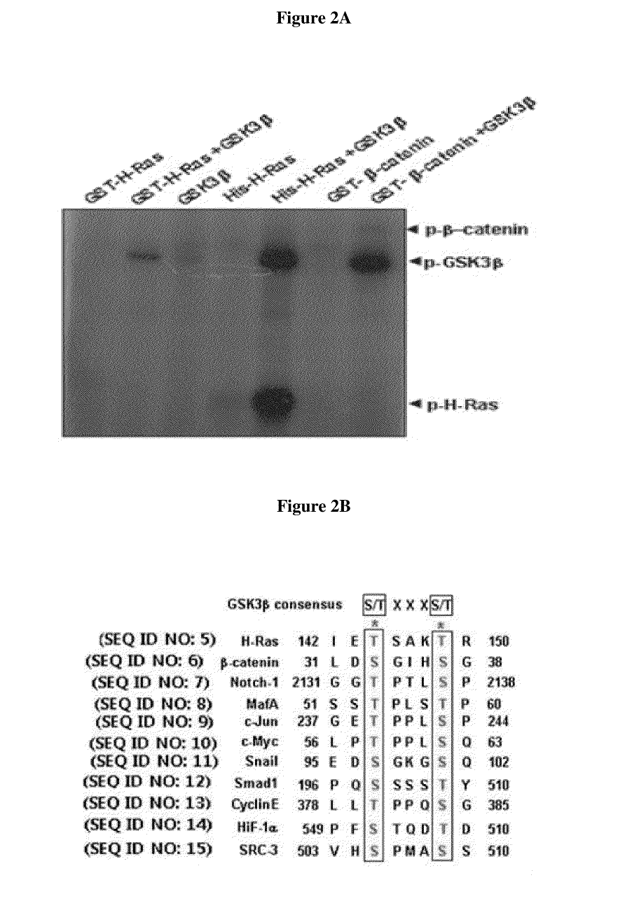

His-H-Ras protein was expressed in Escherichia coli (E. coli) C41 (BL21 [DE3] derivative cells) and purified with Ni-nitrilotriacetic (NTA)-Sepharose resin and Glutathione S-transferase (GST)-H-Ras and GST-β-catenin was purified with glutathione-agarose beads. In vitro kinase analyses were performed with 2 μg of purified His-H-Ras, GST-H-Ras or 1 μg GST-β-catenin together with 200 ng of recombinant human active Gsk3β protein in 20 μl kinase buffer [50 mM Tris-Cl (pH 7.5), 10 mM MgCl₂, 1 mM dithiothreitol (DTT)] containing 10 mM ATP and 10 μCi of [ [symbol] -₃₂P]ATP for 4 hours at 30° C. Reactions were stopped by adding 5×SDS sample buffer followed by heating at 95° C. for 5 minutes. The samples were subjected to 10% SDS-PAGE, and phosphorylated protein images were obtained by autoradiography of the dried gels. In vitro kinase assay using purified recombinant GST-H-Ras, His-H-Ras, GST-β-catenin and Gsk3β proteins proved that H-Ras was strongly phosphorylated by Gsk3β when using the β-catenin, also phosphorylated by Gsk3β, as a positive control. Arrowheads indicate the positions of phosphorylated β-catenin (p-β-catenin), autophosphorylated Gsk3β• (pGsk3β), •• and phosphorylated H-Ras (p-H-Ras) (FIG. 2A). We found that the Thr-144 and Thr-148 residues of H-Ras (₁₄₄TSAKT₁₄₈) are located in a Gsk3β phosphorylation consensus motif “S/TXXXS/T” and sequence alignment of Gsk3β substrates at the regions near the phosphorylation sites indicated that the amino acids are also conserved in H-Ras (FIG. 2B).

### Example 3

### Confirm the Phosphorylation Sites of H-Ras by Gsk3β•

The phosphorylation site of H-Ras by Gsk3β was revealed by Tandem LC/MS-MS analyses. 4 μg of purified His-H-Ras together with 400 ng of recombinant human active Gsk3β protein in 40 μl kinase buffer [50 mM Tris-Cl (pH 7.5), 10 mM MgCl₂, 1 mM dithiothreitol (DTT)] containing 20 mM ATP for 4 hours at 30° C. Reactions were stopped by adding 5×SDS sample buffer followed by heating at 95° C. for 5 minutes. The samples were separated by 10% SDS-PAGE, and H-Ras protein bands were excised for in-gel digestion with 25 ng/ml trypsin and the phosphorylation of H-Ras at Thr-144 and Thr-148 was analyzed by nanoelectrospray liquid chromatography tandem mass spectrometry (LC-MS-MS).

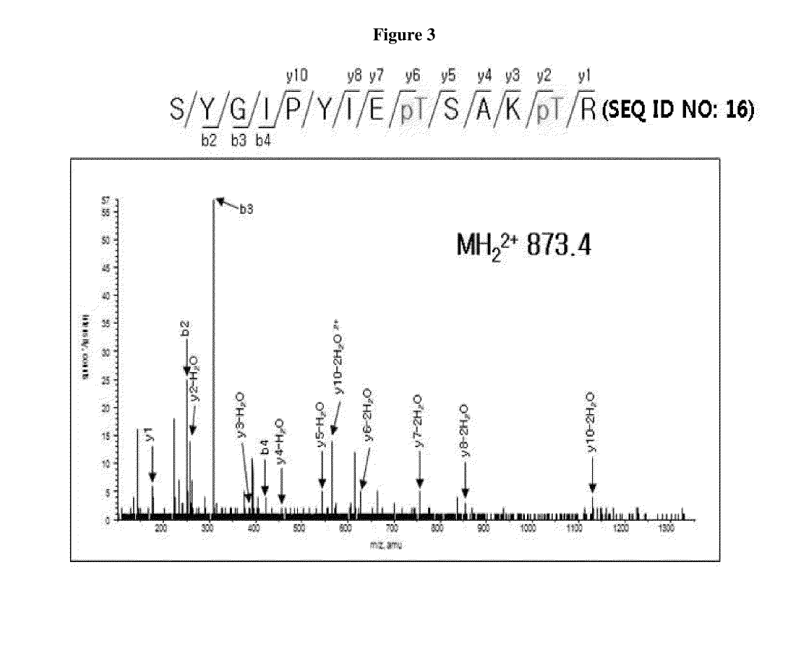

Phosphorylation of H-Ras (SEQ ID NO:16) at Thr-144 and Thr-148 by Gsk3β was identified using Tandem LC/MS-MS analyses of the phosphorylated H-Ras band (FIG. 3).

### Example 4

### The Role of Phosphorylation of H-Ras Via Gsk3β in •β-TrCP-Mediated Ras Polyubiquitination

To examine the importance of Thr-144 and Thr-148 phosphorylation in stability regulation of H-Ras, both phospho-deficient (T144A/T148A) and—mimetic (T144E/T148E) mutants were generated in pcDNA3.1-H-Ras [symbol] pMT3-H-Ras^{L61} and pEGFP-C3-H-Ras. H-Ras mutations of the phosphorylation sites (T144/148A, T144/148E) were generated by PCR-based mutagenesis. Mutations were confirmed by nucleotide sequencing analyses.

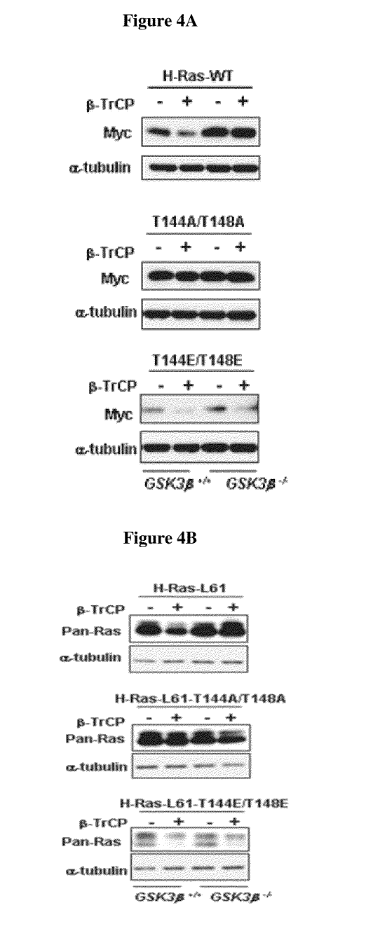

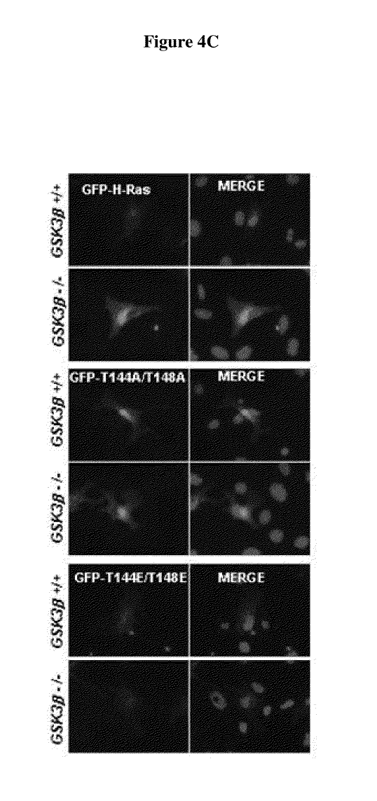

First, we examined the effects of •β-TrCP overexpression on phosphorylation deficient or mimetic mutant H-Ras proteins in Gsk3β+/+ and Gsk3β−/− cells (FIG. 4A). Gsk3β+/+ or Gsk3β−/− cells were transfected with pcDNA3.1-H-Ras (Wt, T144/148A, T144/148E) for 24 hours. WCEs were immunoblotted with anti-Myc, or anti-α•-tubulin antibody. The β-TrCP-mediated degradation of Wt H-Ras was observed only in the Gsk3β+/+ cells, which has Gsk3β that phosphorylates the H-Ras; and not in the Gsk3β−/− cells. The phospho-deficient mutant T144A/T148A H-Ras was not regulated by •β-TrCP in both Gsk3β+/+ and Gsk3β−/− cells. In contrast, the phosphor-mimetic T144E/T148E H-Ras expressed poorly and was further degraded by β•-TrCP transfection in both Gsk3β+/+ and Gsk3β−/− cells (FIG. 4A).

We next examined the effect of β-TrCP overexpression of phosphorylation mutants on oncogenic H-Ras proteins pMT3-H-Ras^{L61} (Wt, T144/148A, T144/148E) in Gsk3β+/+ and Gsk3β−/− cells (FIG. 4B). Cells were transfected with pMT3-H-Ras^{L61} (Wt, T144/148A, T144/148E) for 24 hours and subjected to immunoblotting with anti-Pan-Ras or -α-tubulin antibody. Consistent with Wt H-Ras regulation, the β-TrCP-mediated degradation of oncogenic H-Ras was only observed in Gsk3β+/+ cells not in the Gsk3β−/− cells. The •β-TrCP-mediated degradation of phospho-deficient form of oncogenic H-Ras was mostly abolished in both Gsk3β+/+ and Gsk3β−/− cells, but phospho-mimetic form of oncogenic H-Ras was significantly degraded (FIG. 4B).

The current example demonstrates that both Wt and oncogenic H-Ras is regulated by the same mechanism which phosphorylated at Thr-144 and Thr-148 by Gsk3β, and is subjected to degradation.

Moreover, the stability of Wt and phosphorylation mutant H-Ras proteins was also monitored by immunofluorescence analyses of GFP-tagged proteins in Gsk3β+/+ and Gsk3β−/− cells. Gsk3β+/+ or Gsk3β••••• cells were grown on cover slips coated with gelatin and transfected with pEGFP-C3-H-Ras (Wt, T144/148A, T144/148E) for 24 hours. Cells were fixed with 4% paraformaldehyde in PBS at pH 7.4. DAPI was applied to stain nuclei. Cells were imaged using a Radiance 2100 laser scanning confocal microscope operated by Lasersharp 2000 software. The overexpressed GFP-H-Ras was significantly stabilized in the Gsk3β−/− cells compared to that in Gsk3β+/+ cells, the GFP-T144A/T148A H-Ras was stabilized, and the GFPT144E/T148E H-Ras was destabilized regardless of the presence of Gsk3β (FIG. 4C)

### Example 5

### The Role of Wnt/β-Catenin Pathway on H-Ras Phosphorylation Via Gsk313

To confirm that phosphorylation of H-Ras by Gsk3β also affects the polyubiquitination of H-Ras, we tested the stability regulation of Wt and phosphorylation mutants in HEK293 cells.

First, HEK293 cells were transfected with pcDNA3.1-H-Ras(Wt, T144/148A, T144/148E) for 24 hours and then lysed. WCEs were immunoblotted with anti-Myc or -α-tubulin antibody.

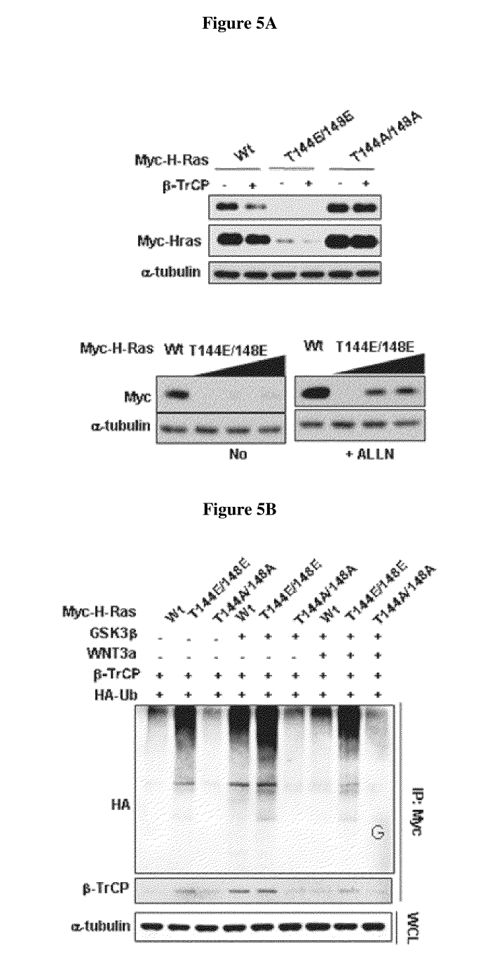

Consistent with the results of Gsk3β+/+ cells, Wt and T144E/T148E H-Ras were degraded by β-TrCP overexpression, and the T144A/T148A H-Ras were not regulated in this way in HEK293 cells (FIG. 5A upper panels).

The protein expression level of the phosphorylation mimetic mutant is too low to be detected due to the severe destabilization; therefore, the proteasomal inhibitor, ALLN was treated to check for the accumulation of phosphor-mimetic form of H-Ras.

HEK293 cells were transfected with pcDNA3.1-H-Ras, pcDNA3.1-H-Ras-T144E/T148E, or pcDNA3.1-H-Ras-T144D/T148D. For indicated cases, ALLN was also administered for 12 hours. WCEs were immunoblotted with anti-Myc or -α-tubulin antibody (FIG. 5A lower panels). Treatment with ALLN increased the level of phosphor-mimetic form of H-Ras.

To identify the role of phosphorylation at Thr-144 and Thr-148 in H-Ras ubiquitination, the degree of polyubiquitination was monitored using H-Ras phosphorylation mutants.

HEK293 cells were transfected the pcDNA3.1-H-Ras, pcDNA3.1-H-Ras-T144E/T148E, or pcDNA3.1-H-Ras-T144A/T148A with vector, pcDNA3.1-Gsk3β-V5, and/or pcDNA3.1-Flag-β-TrCP, together with pCS4-3xHA-Ub, treated with ALLN for 12 hours and/or Wnt3a for 2 hours as indicated, and prepared for IP with anti-Myc antibody. IPs and WCEs were immunoblotted with anti-HA, -β-TrCP or -α-tubulin antibody.

The polyubiquitination and interaction with β-TrCP of Wt H-Ras, increased due to Gsk3β transfection, was blocked by treating cells with recombinant Wnt3a, and this blockage correlated with a reduction of the binding affinity of β-TrCP. Notably, the level of polyubiquitinated phospho-mimetic form of H-Ras in the absence of Gsk3β transfection was almost equivalent to that of Wt H-Ras transfected with Gsk3β, • and the binding affinity of β-TrCP was also strong. However, the phospho-deficient form of H-Ras was not affected by Gsk3β or Wnt3a and its β-TrCP binding affinity was weak.

### Example 6

### Detection of Phosphorylation of H-Ras at Thr-144 and Thr-148 Via Gsk3β with Anti-p-Ras Antibody

To confirm phosphorylation of H-Ras at the Thr-144 and Thr-148 residues in cells, we generated an anti-p-H-Ras polyclonal antibody by immunizing rabbits with keyhole limpet hemocyanin (KLH) carrier protein-conjugated phosphorylated peptide (residues 140-152; PYIEpTSAKpTRQGV) and sacrificed them to obtain the anti-p-Ras antibody.

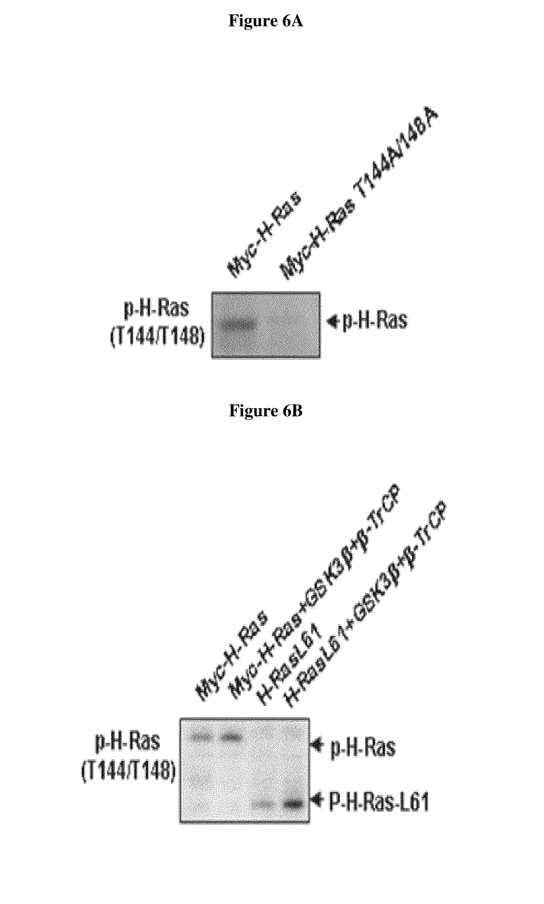

To validate the specificity of this antibody, HEK293 cells were transfected with Wt H-Ras and phospho-deficient T144A/T148A H-Ras, and then WCEs were subjected to immunoblotting. This antibody specifically recognized phosphorylated H-Ras (p-H-Ras) as revealed by a lack of detection of the T144A/T148A H-Ras (FIG. 6A).

Next, we examined whether phosphorylation of Ras increased by Gsk3β or not. HEK293 cells were transfected with pcDNA3.1-H-Ras or pMT3-H-Ras^{L61} and/or pcDNA3.1-Flag-β-TrCP, pcDNA3.1-Gsk3β-V5 and WCEs were immunoblotted with anti-p-Ras antibody. As a result, overexpression of Gsk3β increased the overall levels of Wt and oncogenic form of p-H-Ras (FIG. 6B).

### Example 7

### Inhibition of Cellular Proliferation and Transformation by β-TrCP Through the H-Ras Protein Stability Regulation

To investigate the importance of the phosphorylation and degradation of H-Ras in cellular proliferation and transformation, we monitored the effects of phospho-deficient mutations on proliferation and transformation.

Gsk3β+/+ or Gsk3β−/− cells were transfected with vector, pcDNA3.1-Flag-β-TrCP, or pCS2-MT-Axin together with pMT3-H-Ras^{L61}, or pMT3-HRas^{L61}-T144A/T148A and then seeded into 3 wells of 96-well plates. Viable cell numbers were determined at 0, 24, 48, and 72 hr after seeding via a colorimetric assay using the Cell Counting Kit-8 proliferation assay kit. The results were expressed as mean±S.D. from three independent experiments.

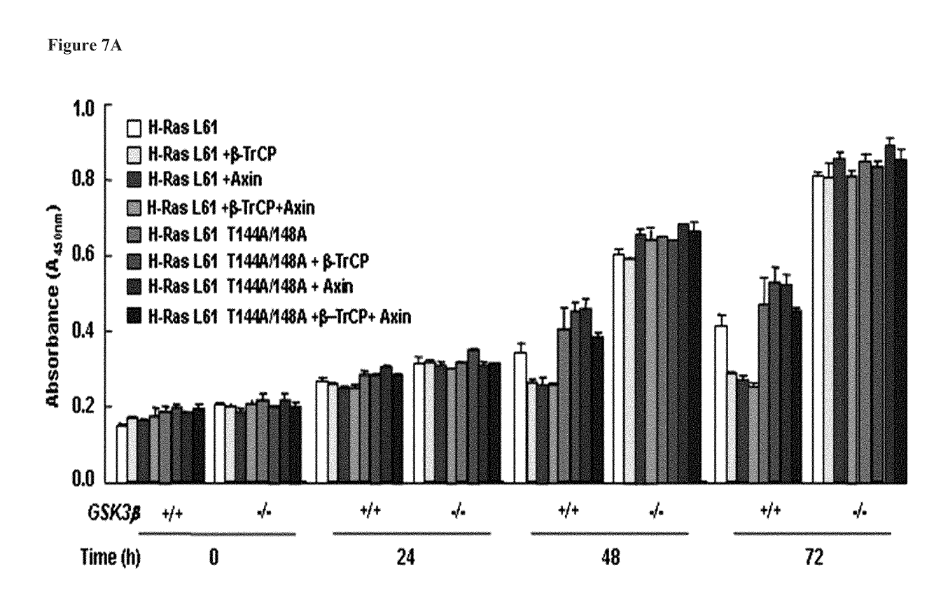

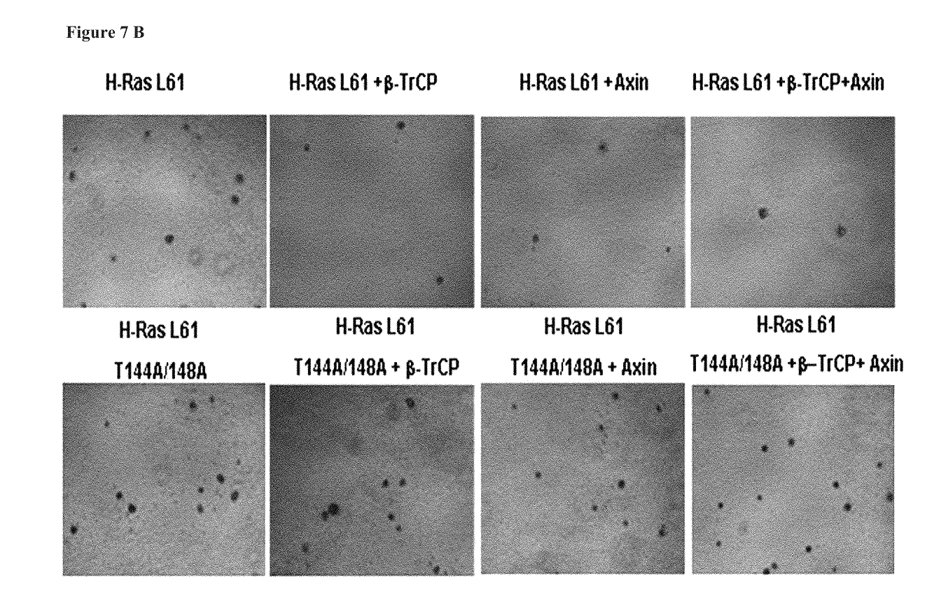

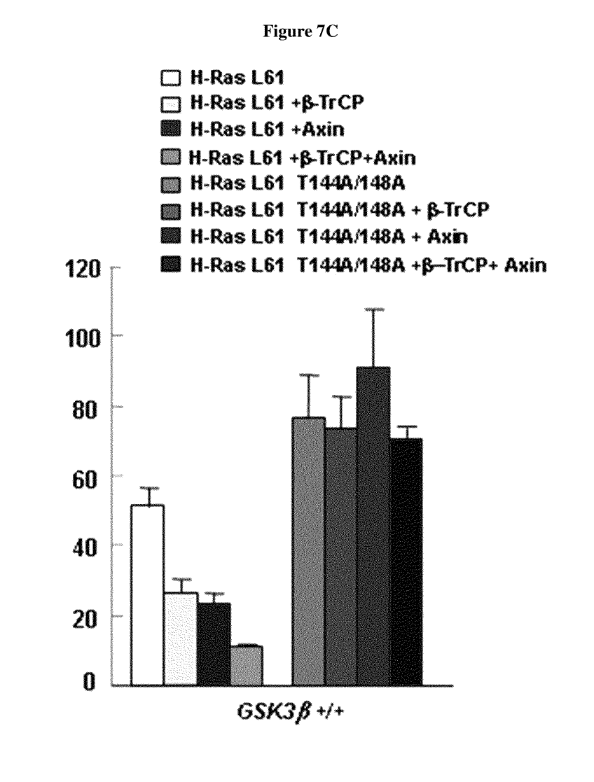

The proliferation of cells transfected with oncogenic H-Ras^{L61} reduced by transfection of β-TrCP and/or Axin in Gsk3β+/+ cells. Proliferation of cells transfected with H-Ras^{L61}-T144A/T148A, however, was not affected by transfection of either β-TrCP and/or Axin in Gsk3β+/+ cells. Therefore, cellular proliferation by either H-Ras^{L61} or H-Ras^{L61}-T144A/T148A was not regulated by β-TrCP and/or Axin in Gsk3β−/− cells (FIG. 7A).

Moreover, under the same experimental condition, anchorage-independent cell growth was monitored by a colony-forming assay of cells in soft agar. Cells (1×10³ cells) were seeded into 3 wells of 96-well plates filled with soft agar, colonies were selected with 800 μgml of G418 contained DMEM for 20 days and media was changed twice a week. Colonies were scored after 20 days when the average colony size had reached a certain size. Colony numbers were counted using Image pro 5.1 Media cybernetics program.

Oncogenic H-Ras^{L61}-induced anchorage-independent cell growth was critically reduced by β-TrCP and/or Axin transfection in Gsk3β+/+; however, these effects were abolished in Gsk3β−/− cells. These inhibitory effects was also blocked in both cells expressing phosphor-decifient mutant H-Ras^{L61}-T144A/T148A. Overall, phosphorylation of H-Ras at Thr-144 and Thr-148 by Gsk3β is essential for the suppression of cellular proliferation and transformation induced by oncogenic H-Ras.

### Example 8

### Polyubiquitin-Dependent Proteasomal Degradation of H-, K-, N-Ras by β-TrCP

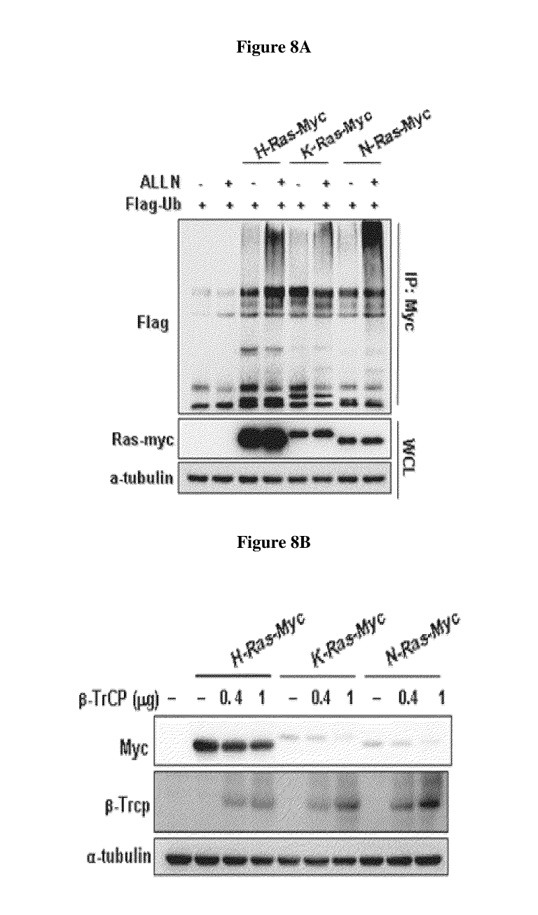

To examine the possibility that K- and N-Ras proteins, similar to H-Ras, were subjected to polyubiquitination by proteasomal degradation, HEK293 cells were transfected with pcDNA3.1-H-Ras, pcDNA3.1-K-Ras, or pcDNA3.1-N-Ras with pCS4-3xFlag-Ub. Cells were treated with ALLN followed by IP with anti-Myc antibody and then immunoblotted with anti-Myc, -α-tubulin, or -Flag antibody. The accumulation of polyubiquitin-conjugated K- and N-Ras proteins following ALLN treatment was confirmed. Therefore, we confirmed that H-, K-, N-Ras protein stability is regulated by polyubiquitin-dependent proteasomal degradation (FIG. 8A).

Next, we checked the effects of β-TrCP on the stability of K- and N-Ras by overexpression in a dose-dependent manner. HEK293 cells were transfected with pcDNA3.1-H-, K- and N-Ras and/or pcDNA3.1-Flag-β-TrCP. WCEs were immunoblotted with anti-Myc, -α-tubulin, or -β-TrCP antibody. Dose-dependent overexpression of β-TrCP destabilized H-, K-, and N-Ras protein levels in a dosage-dependent manner (FIG. 8B).

### Example 9

### Protein Stability Regulation of H-, K-, N-Ras by Gsk3β•

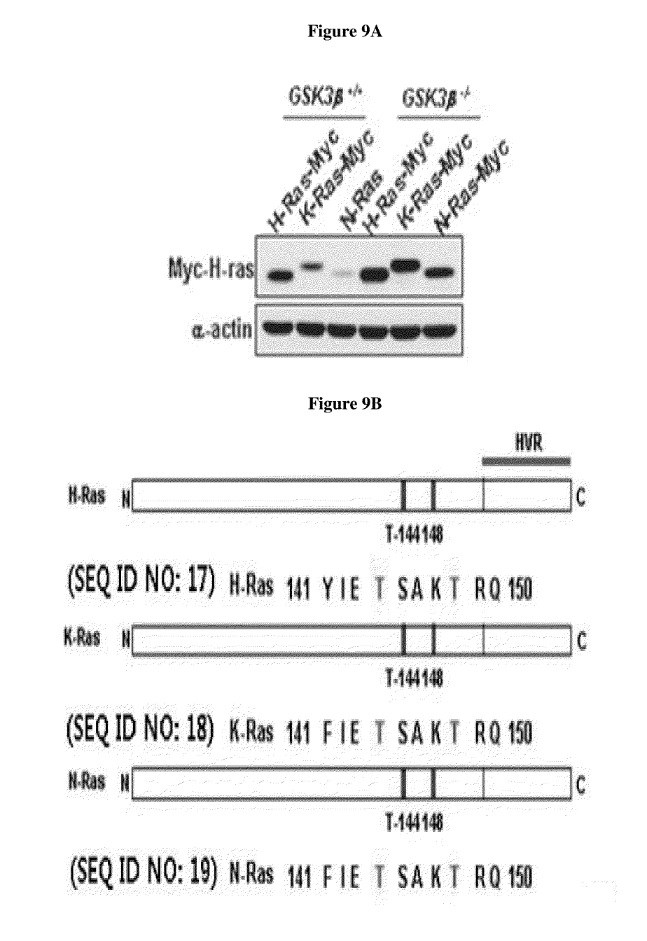

We confirmed that H-, K-, N-Ras protein levels are regulated by polyubiquitin-dependent proteasomal degradation by β-TrCP. Next, we checked that Ras could be phosphorylated and subject to degradation by Gsk3β. Gsk3β+/+ or Gsk3β−/− cells were transfected with pcDNA3.1-H-, K- and N-Ras. WCEs were immunoblotted with anti-Myc and -α-tubulin antibody. The levels of K- and N-Ras, similar to H-Ras significantly increased in Gsk3β−/− cells compared to Gsk3β+/+ cells (FIG. 9A).

K-(SEQ ID NO:18) and N-Ras (SEQ ID NO:19) proteins, similar to H-Ras (SEQ ID NO:17), were subjected to polyubiquitin-dependent proteasomal degradation by β-TrCP, as we checked the Gsk3β phosphorylation consensus motif in K- and N-Ras which are located in H-Ras. Gsk3β phosphorylation consensus motif “S/TXXXS/T” is also conserved in other Ras isotypes, and indicates that Ras degradation via Gsk3β-mediated phosphorylation at Thr-144 and -148 is essential for suppression of cellular proliferation and transformation caused by Ras mutations.

Epidermal growth factor receptor (EGFR) monoclonal antibodies (mAb) such as Avastin, Erbitux and Rituximab are used widely to treat colorectal cancer (mCRC) patients; therefore, it has been anticipated to become engines for growth in the anti-cancer drug market. However, some patients show resistance to the EGFR mAbs due to boring K-Ras mutations. Oncogenic mutations in genes encoding K-Ras proteins lead to their constitutive activation. This phenomenon leads to constitutive, growth-factor receptor independent activation of K-Ras downstream signaling in tumor cells. For this reason, blockade of EGFR activation may have little effect on the activation of K-Ras and its downstream signaling in cancer cells with mutated K-RAS. This phenomenon is becoming noticed due to the high frequency, 4 out of 10, of the K-Ras mutation in colon cancer patients and this mutation increases with tumor progression.

Colorectal cancer is one of the leading causes of cancer-related deaths and unsuccessful anti-cancer therapies, increasing the need for the novel anti-cancer drug development. Under these circumstances the novel mechanism for Ras destabilization, both Wt and oncogenic Ras forms, in our current study offers a potential breakthrough to overcome the unsuccessful anti-cancer therapies targeting Ras, and may be useful for the development of anti-cancer drugs that directly target Ras proteins.

What is claimed is:

1. A method of analyzing the level of Ras protein, comprising examining whether or not phosphorylation of Ras occurs at Thr-144 and Thr-148 sites of Ras.

2. The method of claim 1, wherein whether or not the phosphorylation occurs is examined through an antigen-antibody reaction using an antibody specifically recognizing phosphate groups of Thr-144 and Thr-148 of Ras.

3. The method of claim 2, wherein the antibody is an antibody against a Ras peptide including amino acids 140-152 of Ras, in which Thr-144 and Thr-148 residues are phosphorylated.

4. A composition for diagnosing cancer, comprising an antibody specifically recognizing phosphate groups of Thr-144 and Thr-148 of Ras.

5. The composition of claim 4, wherein the antibody is an antibody against a Ras peptide including amino acids 140-152 of Ras, in which Thr-144 and Thr-148 residues are phosphorylated.

6. A kit for diagnosing cancer, comprising an antibody specifically recognizing phosphate groups of Thr-144 and Thr-148 of Ras.

7. The kit of claim 6, wherein the antibody is an antibody against a Ras peptide including amino acids 140-152 of Ras, in which Thr-144 and Thr-148 residues are phosphorylated.

8. A method of screening for an anti-cancer drug, comprising contacting Ras with a candidate material, examining whether or not phosphorylation of Ras occurs at Thr-144 and Thr-148 sites of Ras, and determining whether the candidate material inhibits or stimulates the phosphorylation.

9. The method of claim 8, wherein whether or not the phosphorylation occurs is examined through an antigen-antibody reaction using an antibody specifically recognizing phosphate groups of Thr-144 and Thr-148 of Ras.

10. The method of claim 9, wherein the antibody is an antibody against a Ras peptide including amino acids 140-152 of Ras, in which Thr-144 and Thr-148 residues are phosphorylated.

11. A method of screening for an anti-cancer drug, comprising treating a cell or an animal, excluding humans, with a candidate material; examining whether or not phosphorylation of Ras occurs at Thr-144 and Thr-148 sites of Ras; and determining whether the candidate material inhibits or stimulates phosphorylation.

12. The method of claim 11, wherein whether or not phosphorylation occurs is examined through an antigen-antibody reaction using an antibody specifically recognizing phosphate groups of Thr-144 and Thr-148 of Ras.

13. The method of claim 12, wherein the antibody is an antibody against a Ras peptide including amino acids 140-152 of Ras, in which Thr-144 and Thr-148 residues are phosphorylated.

14. A method of verifying the effectiveness of an anti-cancer drug, comprising treating an animal, excluding humans, with an anti-cancer drug; examining whether or not phosphorylation of Ras occurs at Thr-144 and Thr-148 sites of Ras; and determining whether the anti-cancer drug inhibits or stimulates the phosphorylation.

15. An antibody specifically recognizing phosphate groups of Thr-144 and Thr-148 of Ras.

16. The antibody of claim 15, wherein the antibody is an antibody against a Ras peptide including amino acids 140-152 of Ras, in which Thr-144 and Thr-148 residues are phosphorylated.


SEQUENCE LISTING (19 sequences)

SEQ ID NO: 1 - protein, 13 aa, Homo sapiens
  MOD_RES (5)..(5): PHOSPHORYLATION
PYIETSAKTRQGV

SEQ ID NO: 2 - RNA, 19 nt, Artificial Sequence
  Primer
cacugauuauaccucuagu

SEQ ID NO: 3 - RNA, 19 nt, Artificial Sequence
  Primer
cacuguaacauaguccgau

SEQ ID NO: 4 - DNA, 21 bp, Artificial Sequence
  This is a combined DNA/RNA sequence
guucagcguguccggcgagtt

SEQ ID NO: 5 - protein, 8 aa, Homo sapiens
IETSAKTR

SEQ ID NO: 6 - protein, 8 aa, Homo sapiens
LDSGIHSG

SEQ ID NO: 7 - protein, 8 aa, Homo sapiens
GGTPTLSP

SEQ ID NO: 8 - protein, 8 aa, Homo sapiens
SSTPLSTP

SEQ ID NO: 9 - protein, 8 aa, Homo sapiens
GETPPLSP

SEQ ID NO: 10 - protein, 8 aa, Homo sapiens
LPTPPLSQ

SEQ ID NO: 11 - protein, 8 aa, Homo sapiens
EDSGKGSQ

SEQ ID NO: 12 - protein, 8 aa, Homo sapiens
PQSSSSTY

SEQ ID NO: 13 - protein, 8 aa, Homo sapiens
LLTPPQSG

SEQ ID NO: 14 - protein, 8 aa, Homo sapiens
PFSTQDTD

SEQ ID NO: 15 - protein, 8 aa, Homo sapiens
VHSPMASS

SEQ ID NO: 16 - protein, 14 aa, Homo sapiens
SYGIPYIETSAKTR

SEQ ID NO: 17 - protein, 10 aa, Homo sapiens
YIETSAKTRQ

SEQ ID NO: 18 - protein, 10 aa, Homo sapiens
FIETSAKTRQ

SEQ ID NO: 19 - protein, 10 aa, Homo sapiens
FIETSAKTRQ